(19) United States
(12) Patent Application Publication
(10) Pub. No.: US 2004/0133279 A1
(43) Pub. Date: Jul. 8, 2004

(54) Surgical implants for use as spinal spacers

(76) Inventors: David J. Krueger, Cedar Park, TX (US); Margaret E. Mitchell, Cedar Park, TX (US); James M. Freid, Round Rock, TX (US)

Correspondence Address: ERIC B. MEYERTONS, Esq., MEYERTONS, HOOD, KIVLIN, KOWERT & GOETZEL, P.C., P.O. BOX 398, AUSTIN, TX, 78767-0398, US

(21) Appl. No.: 10/337,219

(22) Filed: Jan. 6, 2003

(51) Int. Cl. A61F 2/44

(52) U.S. Cl. 623/17.16

(57) ABSTRACT

A spinal implant is provided which maintains intervertebral spacing and stability within the spine. In some embodiments, two or more spinal implants may interlock to form a spinal stabilization system. Spinal implants may interlock using protrusions, indentations, teeth, and/or grooves. In an embodiment, an opening may be positioned in the spinal implant to fuse the spinal implant to surrounding bone tissue. Bone growth through the opening may be increased by using a removable bone growth stimulating insert in the opening. A spinal implant may be constructed of biocompatible material, for example, bone, metal, and/or polymers.

### BACKGROUND OF THE INVENTION

1. Field of the Invention

The present invention generally relates to the field of spinal implants. More particularly, certain embodiments of this invention relate to an allograft implant for stabilizing a portion of the spine (e.g., cervical portion).

2. Description of Related Art

An intervertebral disc may degenerate. Degeneration may be caused by trauma, disease, and/or aging. An intervertebral disc that becomes degenerated may have to be partially or fully removed from a spinal column. Partial or full removal of an intervertebral disc may destabilize the spinal column. Destabilization of a spinal column may result in alteration of a natural separation distance between adjacent vertebrae. Maintaining the natural separation between vertebrae may prevent pressure from being applied to nerves that pass between vertebral bodies. Excessive pressure applied to the nerves may cause pain and/or nerve damage. During a spinal fixation procedure, a spinal implant may be inserted within a space created by the removal or partial removal of an intervertebral disc between adjacent vertebrae. The spinal implant may maintain the height of the spine and restore stability to the spine. Bone growth through or around the implant may fuse the adjacent vertebrae.

A spinal implant may be inserted during a spinal fixation procedure using an anterior, lateral, or posterior spinal approach. In some situations, an anterior approach may result in an easier approach, less muscle and tissue damage, and less bone removal than other approaches.

A discectomy may be performed to remove or partially remove a defective or damaged intervertebral disc. The discectomy creates a disc space for a spinal implant. The amount of removed disc material may correspond to the size and type of a spinal implant to be inserted. After a discectomy, a spinal implant may be inserted into the disc space. One or more spinal implants may be inserted between a pair of vertebrae. Spinal implants may be inserted into disc spaces prepared between more than one pair of vertebrae during a spinal fusion procedure.

Spinal surgery may be complex due in part to the proximity of delicate soft tissue such as the spinal cord and/or vascular structures. Preparation instruments and spinal implants may need to be carefully inserted to avoid damage to soft tissue. Alignment and spacing of a spinal implant that is to be inserted into a patient may be determined before surgery. Achieving the predetermined alignment and spacing during surgery may be important to achieve optimal fusion of adjacent vertebrae.

Bone graft and/or bone implants may be used to promote bone growth that will fuse vertebrae together. Bone graft may be autogenic bone, allogenic bone, synthetic material, xenogenic bone or combinations thereof. Autogenic bone is bone obtained from another location of a patient. Allogenic bone is bone derived from the same species as the patient. Xenogenic bone is bone derived from a species other than that of the patient. Implants may be formed of metal, polymers, ceramics, autogenic bone, allogenic bone, xenogenic bone, or combinations thereof.

Posterior lumbar interbody fusion using cylindrical, smooth bone grafts as spacers was reported by several authors by mid-1950. By 1985, threaded cylindrical bone dowels were reported. Threaded dowels eliminated the need to impact dowels in position during insertion. To fuse vertebrae with a threaded dowel, an opening was drilled and tapped into end plates of adjacent vertebrae. The bone dowel was then screwed into the opening.

U.S. Pat. No. 5,814,084 to Grivas et al., which is incorporated by reference as if fully set forth herein, describes diaphysial cortical bone dowels. The dowels are obtained from transverse plugs across the diaphysis of long bones. The natural intramedullary canal of the source bone may form a cavity through the dowel perpendicular to the length of the dowel.

U.S. Pat. No. 6,025,538 to Yaccarino, III, which is incorporated by reference as if fully set forth herein, describes a composite allograft bone device. A first bone component is formed with a plurality of grooves. A second bone component is formed with a plurality of protrusions that mate with the grooves of the first bone component. A pin positioned at an oblique angle through the bone components joins the components together to form the composite allograft bone device.

U.S. Pat. No. 6,143,033 to Paul et al., which is incorporated by reference as if fully set forth herein, describes an allogenic intervertebral implant. The intervertebral implant is an annular plug that conforms in size and shape to end plates of adjacent vertebrae. Top and bottom surfaces of the implant have teeth to resist expulsion and to provide initial stability.

A surface of an implant may be treated to inhibit expulsion and to provide stability. Surface treatment may include, but is not limited to, sanding, forming grooves within a surface, shot peening processes, electrical discharge processes, and/or embedding of hard particles within a surface. For example, a method for embedding sharp hardened particles in a metal surface is described in U.S. Pat. No. 4,768,787 issued to Shira; a method for forming a frictional surface within a metal surface using an electrical discharge process is described in U.S. Pat. No. 4,964,641 issued to Miesch et al.; and a shot peening process for forming a textured surface is described in U.S. Pat. No. 5,526,664 to Vetter, all of which are incorporated by reference as if fully set forth herein.

### SUMMARY OF THE INVENTION

A spinal implant may be used to promote fusion of adjacent vertebrae in, e.g., a cervical region of a spine. In an embodiment, the implant may be used in conjunction with a spinal stabilization device such as a bone plate or rod-and-fastener stabilization system. In an embodiment, the implant may establish a desired separation distance between vertebrae and promote bone growth between adjacent vertebrae to fuse the vertebrae together.

Implants may be inserted into a patient using an instrumentation set. Instruments in the instrumentation set may include, but are not limited to, distractors, chisels, and implant inserters.

Implants may be constructed of any biocompatible materials sufficiently strong to maintain spinal distraction including, but not limited to, autograft bone, allograft bone, xenograft metals, carbon fiber materials, ceramics and/or polymers. An implant, or a portion of an implant, may be made of a bio-absorbable material. For example, an implant may be made of a polyanhydride, an alpha polyester, and/or a polylactic acid-polyglycolic acid copolymer. In some embodiments, an implant may contain a bone growth stimulating material to promote spinal fusion. For example, an opening or openings in a spinal implant may be contain bone graft or a synthetic bone graft substitute.

A spinal implant may have a polygonal shape. The implant may include an opening that extends through a height of the implant. The opening may have a regular or irregular shape. Surfaces of the implant that will contact vertebrae may have texturing. The texturing may, in some embodiments, be a series of ridges.

Some embodiments of an implant may be constructed from allogenic bone, such as cortical bone from a femur, tibia, or other large bone. An implant may be constructed from a single section of bone. Alternatively, an implant may be constructed from two or more sections of bone. The sections may be fastened, pinned, glued, or otherwise affixed together. In some embodiments, a spinal implant may be constructed from two or more layers of a biocompatible material.

A spinal implant may be used individually or in a stacked arrangement. In an embodiment, a first spinal implant may be coupled to a second spinal implant. The coupling may be achieved by connecting a protrusion on the first implant to an indentation on the second implant. In some embodiments, protrusions on a surface of a first implant will interlock with indentations on a second implant. In an embodiment, implants may be stacked and connected to form an initial spinal implant of a desired height. Implants may also be stacked and connected to add spinal implants to an existing implant.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a top view of an embodiment of a spinal implant.

FIG. 2 depicts a top view of an embodiment of a spinal implant.

FIG. 3 depicts a top view of an embodiment of a spinal implant.

FIG. 4 depicts a top view of an embodiment of a spinal implant.

FIG. 5 depicts a perspective view of an embodiment of a spinal implant.

FIG. 6 depicts a side view of an embodiment of a spinal implant.

FIG. 7 depicts a perspective view of an embodiment of a spinal implant having a groove for engaging an insertion device.

FIG. 8 depicts a top view of an embodiment of a spinal implant.

FIG. 9 depicts a perspective view of an embodiment of a spinal implant.

FIG. 10 depicts a perspective view of an embodiment of a spinal implant.

FIG. 11 depicts a top view of an embodiment of a spinal implant.

FIG. 12 depicts a perspective view of an embodiment of a pin.

FIG. 13 depicts a view of an embodiment of a spinal implant inserted into an intervertebral space.

FIG. 14 depicts a top view of an embodiment of a spinal implant.

FIG. 15 depicts a perspective view of an embodiment of a spinal implant.

FIG. 16 depicts a top view of an embodiment of a spinal implant.

FIG. 17 depicts a top view of an embodiment of a spinal implant.

FIG. 18 depicts a perspective view of an embodiment of a spinal implant.

FIG. 19 depicts a perspective view of an embodiment of a spinal implant inserted into an intervertebral space.

FIG. 20 depicts a side view of a spinal implant.

While the invention is susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but on the contrary, the intention is to cover all modifications, equivalents, and alternatives falling within the spirit and scope of the present invention as defined by the appended claims.

### DETAILED DESCRIPTION OF THE EMBODIMENTS

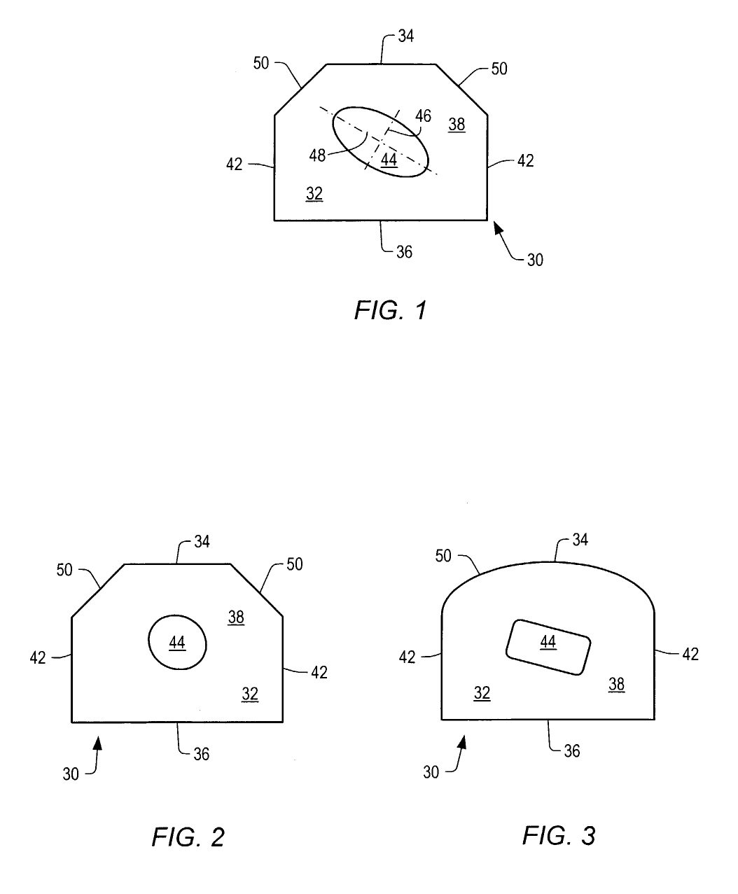

Referring to the figures, FIGS. 1-11 and 13-20 show embodiments of spinal implant 30. Spinal implant 30 may be used to establish a desired separation distance between vertebrae during a procedure that stabilizes a portion of a spine (e.g., cervical, lumbar, thoracic, etc.). In some embodiments, spinal implant 30 may be positioned between cervical vertebrae C1 through C7. For example, spinal implant 30 may be used for surgeries performed on vertebrae C4 and C5. Components of spinal implant 30 may be constructed of a biocompatible material sufficiently strong to maintain vertebral separation. The material may be, but is not limited to, bone, metals, ceramics, polymers, or combinations thereof. Bone growth may fuse spinal implant 30 to adjacent vertebrae.

As shown in FIGS. 1-5, body 32 of spinal implant 30 may have multiple sides, including anterior side 34, posterior side 36, superior side 38, inferior side 40, and lateral sides 42. In some embodiments, body 32 of spinal implant 30 is shaped to substantially conform to the shape of vertebrae between which spinal implant 30 will be placed. The shape of spinal implant 30 may include, but is not limited to, a shape that matches an angle of lordosis, a shape such that superior side 38 and/or inferior side 40 of body 32 is convex, and/or shaped corners on a side of body 32. These features may be combined or used individually to promote a better fit of spinal implant 30 into an intervertebral space.

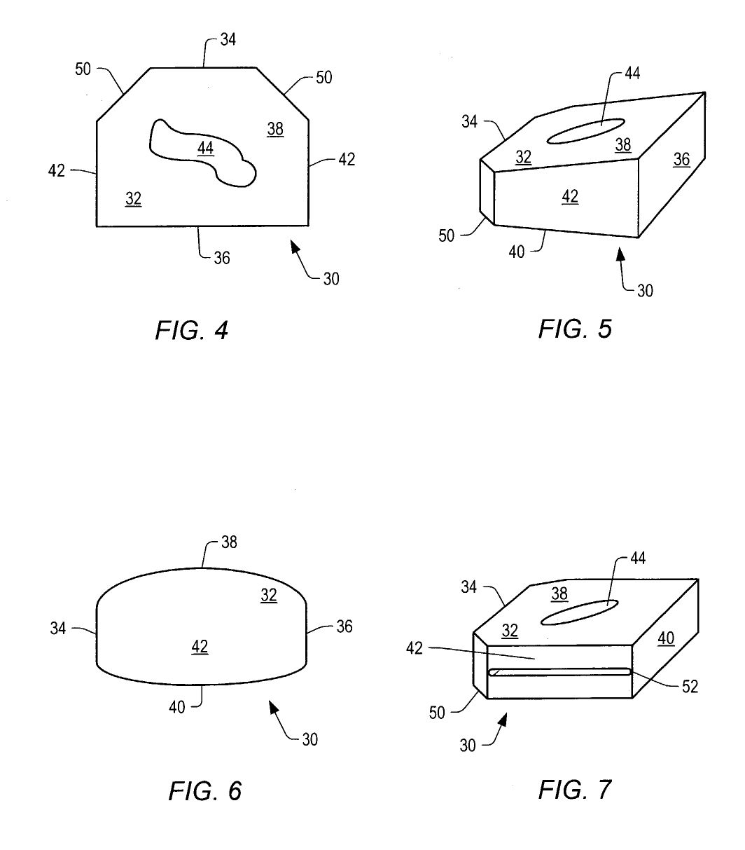

Body 32 of implant 30 may include opening 44. Opening 44 may have, but is not limited to: a substantially oval shape, as shown in FIG. 1; a substantially circular shape, as shown in FIG. 2; a substantially square shape; a substantially rectangular shape, as shown in FIG. 3; or an oblong deviation of any of the above-mentioned shapes. An oblong opening may be defined as an opening that deviates from an opening having a regular shape (such as a square or circle) by having a shape that is elongated along at least one axis. In an embodiment, opening 44 may have an irregular shape, as depicted in FIG. 4.

In some embodiments, opening 44 extends across a face of implant 30. Opening 44 may be oblique to lateral side 42 of body 32, as illustrated in FIG. 1. Opening 44 may extend only partially through the height of implant 30. As shown in FIG. 1, opening 44 may also extend from superior side 38 to inferior side 40 of body 32. In certain embodiments, positioning opening 44 oblique to lateral side 42 of body 32 of spinal implant 30 may enhance the stability of the implant by increasing an amount of support material surrounding opening 44.

Opening 44 may be large to provide a large area for bone growth that will fuse adjacent vertebrae together. Opening 44 may also be sufficiently small so that body 32 of implant 30 provides sufficient strength to prevent fracturing of implant 30 during formation, handling, insertion, and post-insertion use. In some spinal implant embodiments, opening 44 may have first axis 46 and second axis 48. As illustrated in FIG. 1, first axis 46 may be perpendicular to second axis 48. Second axis 48 within opening 44 may be longer than first axis 46 within opening 44. A large opening 44 may be created without significant loss in structural strength of the spinal implant 30 when second axis 48 within opening 44 is longer than first axis 46 within opening 44.

Spinal implant 30 may include indicator 50 that allows an implant installer to distinguish anterior side 34 from posterior side 36. Indicator 50 may be particularly important if the spinal implant 30 is angled to provide a desired lordotic angle. Indicator 50 may be, but is not limited to, a structural indicator such as a rounded corner on a side, a chamfered corner on a side, a notch on a side, and/or a groove on a side. FIG. 1 depicts an embodiment of spinal implant 30 in which indicator 50 includes chamfered comers on anterior side 34 of body 32. FIG. 3 depicts an embodiment in which indicators 50 are rounded comers on anterior side 34 of body 32. In some embodiments, indicators 50 may be indicia that are printed, etched, or otherwise marked on a surface (e.g., posterior side 36) of spinal implant 30.

Body 32 of spinal implant 30 may be shaped to provide lordosis. FIG. 5 shows spinal implant 30 that may provide lordosis. Spinal implant 30 may include indicator 50 and/or opening 44 on a side of the spinal implant. In some embodiments, a height of posterior side 36 of body 32 is greater than a height of anterior side 34 of the body. A lordotic angle provided by spinal implant 30 may range from about 1° to about 12°. In certain embodiments, a lordotic angle provided by spinal implant 30 may range from about 4° to about 8°. Pre-surgery imaging of the vertebrae to receive spinal implant 30 may allow an estimate of a lordotic angle needed for the spinal implant. Spinal implants 30 having lordotic angles slightly less than, substantially equal to, or slightly more than the estimated lordotic angle may also be used.

In some implant embodiments, the body of a spinal implant may have a convex superior side and/or convex inferior side that conforms to a shape of a vertebral surface. FIG. 6 shows biconvex spinal implant 30, in which superior side 38 and inferior side 40 of body 32 are convex. Spinal implant 30 may have anterior side 34, posterior side 36, and lateral sides 42. In some embodiments, spinal implant 30 may include a substantially flat side or a concave side.

FIG. 7 illustrates an embodiment of spinal implant 30 in which body 32 includes groove or slot 52 on lateral side 42. Groove or slot 52 may be used for attachment of an insertion device to body 32 for placement of spinal implant 30 between vertebrae. In some embodiments, spinal implant 30 may include opening 44 through superior side 38. Opening 44 may provide a large area for bone growth that will fuse adjacent vertebrae together. Indicator 50 of spinal implant 30 may include chamfered comers. Indicator 50 may distinguish between anterior side 34 and posterior side 36.

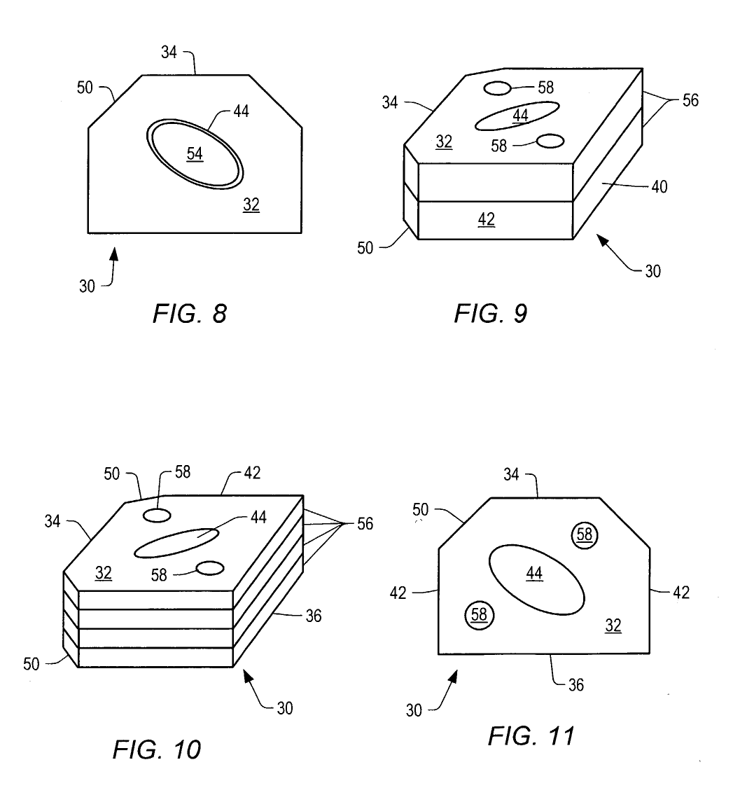

Inducing bone growth around and through a spinal device may promote success of spinal implantation surgery. Bone growth through opening 44 of body 32 can be induced by using an osteogenic material for spinal implant 30. An osteogenic material is generally defined as a material that promotes formation of bone tissue. Osteogenic materials may include, but are not limited to, bone graft, such as cancellous bone; bone growth promoting substances, such as growth hormone; and/or synthetic bone graft substitute. As shown in FIG. 8, spinal implant 30 may be constructed with removable insert 54. Removable insert 54 may include osteogenic material and/or bone growth promoting substances (e.g., growth hormone). Insert 54 may be removed at the discretion of a physician implanting spinal implant 30. In some embodiments, insert 54 may include two or more osteogenic materials. For example, cancellous bone may be used in conjunction with growth hormone. In some embodiments, insert 54 may be positioned within opening 44. Spinal implant 30 may also include chamfered comers as indicator 50 on anterior side 34 of body 32.

To increase the bone growth stimulating properties of bone used in spinal implants 30, the bone may be demineralized. Dimineralizing bone may expose protein in the bone. By exposing protein on body 32 of spinal implant 30, the growth of bone near spinal implant 30 may be substantially increased. Bone used in spinal implant 30 may, in certain embodiments, be partially or fully demineralized. Demineralizing bone may be achieved by soaking a section of bone in an acid wash.

Spinal implant 30 may be constructed from various materials. A single material or a combination of materials may be used. Materials used may include, but are not limited to: metals, including titanium and its alloys; autograft bone and allograft bone, such as partial or fully demineralized bone, including cancellous and cortical bone; graphite and pyrolytic carbon; bioceramics, including hydroxyapatite and calcium phosphate based materials, and bioactive glasses, including Bioglass® and NovaBone™; natural and synthetic polymers, including bioabsorbable polymers such as starches, polyglycolides, polylactides, and glycolide-lactide copolymers; and nonbioabsorbable polymers including polymethyl methacrylate, polytetrafluoroethylene, polyurethane, polyaryl ether ketone resins (e.g., PEEK™), polyethylene, and nylon; as well as composite materials such as a material including polymers and ceramic, ceramic and metal, or any other combination of the above-mentioned materials.

In some cases, X-rays may be used to monitor spinal fusion in a patient. Some implant embodiments (e.g., PEEK implants) are substantially transparent to X-rays. X-ray detection of implant 30 formed of X-ray transparent material may be facilitated by including X-ray sensitive material in the implant. For example, tantalum wire (e.g., 1 mm in length) may be inserted into one or more openings of an implant before implantation. In some embodiments, X-ray sensitive material may be located near an anterior end of the implant adjacent to a caudal (or cephalic) surface of the implant. X-ray sensitive material may also be located near a posterior end of the implant adjacent to a cephalic (or caudal) surface of the implant. The use of X-ray sensitive material near anterior/posterior and caudal/cephalic surfaces may allow the position of an implant to be visualized using X-ray imaging.

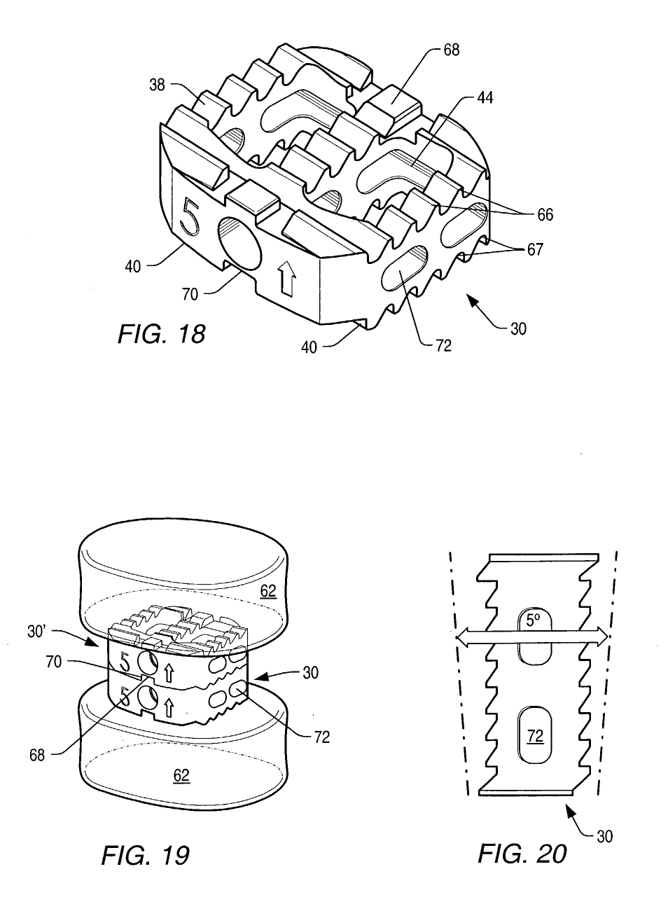

Bone growth that fuses vertebrae together through an implant may be monitored subsequent to an implant insertion procedure. Bone growth in an implant that is not X-ray transparent (e.g., a metallic implant) may be monitored utilizing passages 72 (depicted in FIG. 18). Passages may allow passage of X-rays through the implant so that an X-ray image taken indicates the presence, absence, and/or density of bone in passages of the implant.

In some embodiments, spinal implant 30 may be made from a single section of allogenic bone derived from a donor. However, the amount, integrity, and thickness of bone available from a donor may be limited. As depicted in FIGS. 9 and 10, if available bone pieces are not thick enough to construct body 32 of a desired height with a single section of bone, two or more sections 56 of biocompatible material may be coupled to form body 32 of implant 30. Materials used to form sections 56 of body 32 may be the same or different.

Sections 56 may be joined using a variety of methods including, but not limited to, an adhesive, a fastener (e.g., a screw, a pin, and/or a nail) a dovetail joint, a slot and groove joint, a male/female interface, and/or an interference fit. Adhesives may include: cyanoacrylates; dental resin cements; epoxy-based compounds; dental resin sealants; glass ionomer cements; polymethyl methacrylate; gelatin-resorcinol-formaldehyde glues; inorganic bonding agents such as zinc phosphate, magnesium phosphate, or other phosphate-based cements; and zinc carboxylate.

FIGS. 9, 10, and 11 show embodiments of spinal implant 30 in which body 32 includes fastener openings 58, chamfered comers as indicator 50, anterior side 34, posterior side 36, and lateral sides 42. Fastener openings 58 may extend through first section 56 into second section 56. Fastener openings 58 may receive fasteners that couple sections 56 together. Fasteners may be, but are not limited to, pins, screws, rivets, nails, and/or barbs.

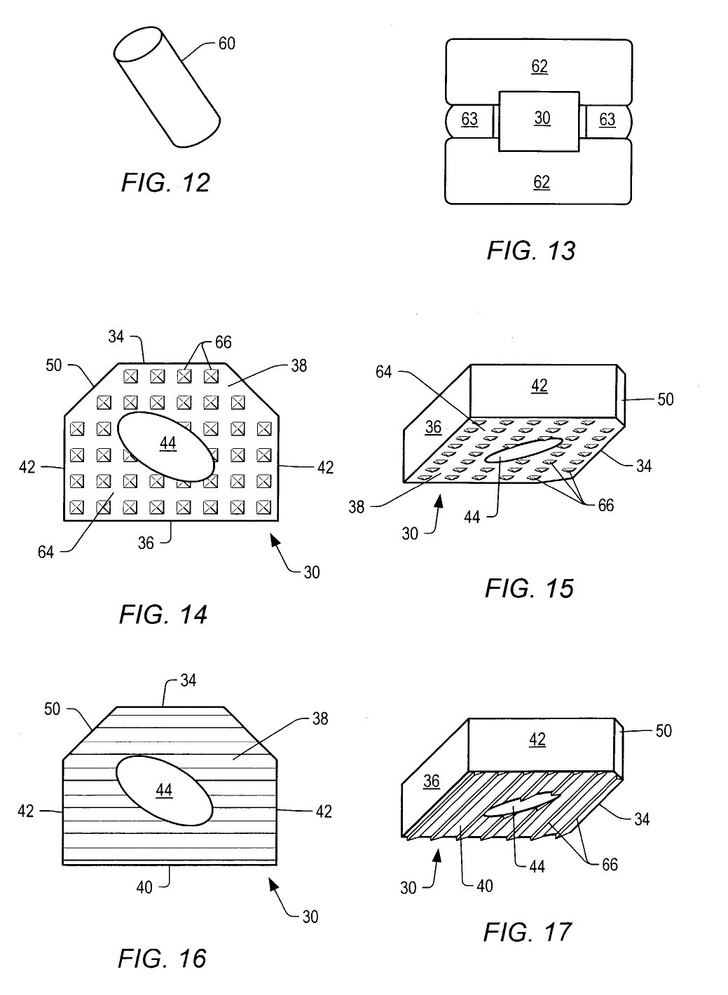

FIG. 12 depicts pin 60 that may couple sections of a spinal implant together. In some embodiments, pin 60 may be press fit into a fastener opening. A fastener opening may be oriented substantially transverse to an opening of the body. The transverse orientation may provide an implant with enhanced stability. In some embodiments, a fastener and a fastener opening may be keyed to inhibit rotation of a first section relative to a second section of a spinal implant. In some embodiments, bone from the same donor may be used to construct one or more sections and at least one pin. In some embodiments, a spinal implant may include sections formed from different materials.

FIG. 13 depicts an embodiment of spinal implant 30 positioned between vertebrae 62 within a cavity formed in disc 63. The cavity may be formed by a discectomy. Material from vertebrae 62 may be removed to enhance a fit of spinal implant 30 between vertebrae 62. Posterior side 36 of spinal implant 30 may be positioned approximately parallel to vertebrae 62.

In some embodiments, outer surface 64 of implant 30 may be formed as a relatively smooth surface. In other embodiments, outer surface 64 may be roughened to create a textured surface, as shown in FIGS. 14-17. Outer surface 64 may be roughened by, but is not limited to being roughened by, scoring, sanding, shot peening, and/or treating the outer surface with an electric discharge. Outer surface 64 may be treated by, but is not limited to being treated by, forming grooves and/or creating teeth on the outer surface and/or embedding hard particles within the outer surface.

In some embodiments, one or more outer surfaces 64 of spinal impant 30 may contain teeth 66. Teeth 66 may have various shapes including, but not limited to, pyramidal (shown in FIGS. 14 and 15) and/or saw-tooth shaped (shown in FIG. 17). In some embodiments, a plurality of grooves may be formed on one or more outer surfaces 64 of spinal implant 30, as shown in FIG. 16. In some alternative embodiments, teeth and grooves may be used together on outer surface 64 of spinal implant 30.

FIGS. 18-20 depict an embodiment of spinal implant 30 for use individually or in a stacked arrangement. Surfaces of spinal implant 30 may engage surfaces of vertebrae 62 and/or surfaces of another spinal implant. Teeth 66 on a superior side of spinal implant 30 may engage grooves 67 on an inferior side of another spinal implant. The teeth and grooves may form a secure fit that inhibits relative movement of spinal implants 30, 30′ in a direction perpendicular to the teeth, as shown in FIG. 19. In addition, protrusions 68 on a surface of spinal implant 30 may engage indentations 70 on spinal implant 30′ to interlock the implants and inhibit relative movement of the implants in a direction parallel to the teeth. For example, protrusions 68 on superior side 38 of spinal implant 30 may connect with indentations 70 on inferior side 40 of spinal implant 30′.

Interlocked spinal implants 30, 30′ may be positioned between vertebrae 62. The option of using more than one spinal implant within the intevertebral space may result in a better fit than a single spinal implant 30 positioned within the intevertebral space. In some embodiments, using more than one spinal implant 30 will establish a desired separation distance between vertebrae 62 and promote bone growth between adjacent vertebrae to fuse the vertebrae together. Using more than one implant 30 may facilitate tailoring of the spinal implant height for each insertion procedure. Some spinal implant embodiments may include multiple openings 72 positioned in body 32 of spinal implant 30. In some embodiments, spinal implant 30 may have a shape that matches an angle of lordosis, as illustrated in FIG. 20.

In an embodiment, spinal implant 30 is formed from metal. Outer surface 64 may be roughened by an electrical discharge process. An electrical discharge may be used to generate a spark between outer surface 64 and an electrode by creating a potential difference between the outer surface 64 and the electrode. The spark produced may remove a portion of outer surface 64 disposed between the electrode and the outer surface to form a cavity in the outer surface. In some embodiments, the electrode is relatively small, resulting in the formation of a small cavity. The electrode may be moved about outer surface 64 to form numerous cavities within the outer surface. In some embodiments, the cavities are substantially pyramidal in shape. Various patterns may be formed within outer surface 64 depending on electrode positioning during the discharge process. In some embodiments, a diamond pattern or a waffle pattern may be formed on one or more outer surfaces 64 of spinal implant 30.

In an embodiment, one or more outer surfaces 64 of spinal implant 30 may be textured by the use of a shot peening method. According to this method, a stream of hardened balls is propelled at a high velocity toward outer surface 64. In some embodiments, the balls are constructed of steel. The stream of balls may be directed toward outer surface 64 to create a pattern on at least a portion of the outer surface. The speed with which the stream is directed toward outer surface 64 may determine texturing of the surface. In some embodiments, the stream is directed such that ridges and valleys are formed in the surface. The ridges and valleys of outer surface 64 may interact with similar formations on vertebrae 62 to provide additional resistance to movement in either a longitudinal direction or a direction perpendicular to a longitudinal axis.

In an embodiment, one or more outer surfaces 64 of spinal implant 30 may be textured by embedding sharp hardened particles in the outer surface using a laser or other high energy source to melt the outer surface in selected areas. A stream of abrasive particles may be directed toward molten areas of outer surface 64 before the outer surface re-solidifies. In this manner, some of the particles may become embedded within the molten surface. In some embodiments, the particles have a number of sharp edges that protrude from outer surface 64 after the particles have been embedded within the outer surface. Any of the above methods of texturing may be used in combination with one or more other methods.

Spinal implant 30 may be used in combination with other devices to provide enhanced stability. In one embodiment, spinal implant 30 is used in combination with pedicle screws. Pedicle screws may be installed in pairs for each vertebral level that requires fixation. A pedicle screw may be inserted through a pre-bored hole at the junction of a superior articular process and transverse process through a pedicle of vertebrae 62.

In an embodiment, spinal implant 30 may be used in combination with a pedicle screw system. The pedicle screw system may stabilize one vertebral level by connecting a second vertebral level using anchoring pedicle screws and connecting rods or plates. The connecting rods or plates may extend between vertebrae 62.

In an embodiment, spinal implant 30 may be used in combination with plates used to couple adjacent vertebrae 62. The plates may vary in size to accommodate variations in spinal anatomy.

In an embodiment, spinal implant 30 may be used in combination with a plate system positioned adjacent to the spine. The plate system may comprise two opposing plates positioned on opposite sides of vertebral spinous processes. The spinal plate system may comprise nut and bolt assemblies positioned in pre-drilled holes in the plates. In some embodiments, the bolts are positioned in the space between the spinous processes of adjacent vertebrae 62. A compressive force may be applied to the lateral sides of the spinous processes by the opposing plates to hold the system to vertebrae 62.

In some embodiments, spinal implants 30 may be constructed from bone harvested from femoral shafts. In certain embodiments, a height of spinal implant 30 may depend on a thickness of the femur. Bone sections cut from the femur may have a length and/or a width in a range from about 10 mm to about 15 mm. In some embodiments, the bone sections may be substantially square in cross section. The size of the femoral shaft from which the bone section is derived may limit the dimensions of the bone sections. For example, some femoral shafts may not be thick enough to form a spinal implant of a desired height. In some embodiments, two or more bone sections cut from a femoral shaft may be coupled together to form a spinal implant of the desired height. In some instances, the bone sections may be coupled together using pins. Surfaces of the bone section may be textured to inhibit movement of a spinal implant from an intervertebral space. In some embodiments, comers of a spinal implant may be chamfered. In addition, the spinal implant may be fenestrated.

Spinal implants made from bone sections may be treated to remove any remaining tissue and/or blood. Implants may be pre-packaged and freeze-dried. A CNC machine may be used to cut pins from matchstick-sized pieces of bone. Alternatively, pins may be cut from the matchstick-sized pieces of bone using a lathe. Use of a CNC machine may increase accuracy when forming the pins. In some embodiments, bone (e.g., bone sections, pins, and/or matchstick-sized pieces) may be freeze-dried prior to completion. Freeze-drying may induce shrinkage in bone; therefore, freeze-drying may occur prior to final machining and/or assembly. Openings may be positioned in sections of bone such that during use the matchstick-sized pieces of bone may couple two sections of bone together. In alternative embodiments, bone pieces (e.g., bars, matchstick-sized pieces) may be treated in liquid baths and then freeze-dried. In some embodiments, openings in the bone sections may be reamed to allow for press fitting the pins in the bone sections.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A spinal implant, comprising:
a first surface and a second surface, wherein the first surface and the second surface are configured to engage vertebrae; and
a protrusion on the first surface of the spinal implant, wherein the protrusion is configured to engage another spinal implant.

2. The spinal implant of claim 1, further comprising an opening extending through the spinal implant.

3. The spinal implant of claim 1, further comprising an indentation on the second surface of the spinal implant.

4. The spinal implant of claim 3, wherein the indentation is configured to engage a portion of another spinal implant.

5. The spinal implant of claim 1, further comprising:
teeth on a surface of the spinal implant; and
grooves on a surface of the spinal implant.

6. The spinal implant of claim 1, further comprising teeth on a surface of the spinal implant wherein at least some of the teeth are configured to engage a portion of another spinal implant.

7. The spinal implant of claim 1, further comprising grooves on a surface of the spinal implant wherein at least some of the grooves are configured to engage a portion of another spinal implant.

8. The spinal implant of claim 1, further comprising an osteogenic insert, wherein the insert is removably occupying the opening.

9. The implant of claim 1, further comprising an X-ray sensitive material in the spinal implant.

10. A spinal implant system, comprising:
a first spinal implant;
a second spinal implant; and
wherein a surface of the first implant is configured interlock with a surface of the second implant to inhibit movement of the second implant relative to the first implant during use.

11. The system of claim 10, further comprising:
an indentation on the first spinal implant;
a protrusion on the surface of the second spinal implant; and
wherein the indentation is configured to engage the protrusion.

12. The system of claim 10, further comprising:
teeth on the first spinal implant;
grooves on the second spinal implant; and
wherein at least some of the teeth inhibit movement of the first spinal implant relative to the second spinal implant.

13. The system of claim 10, further comprising:
a first opening extending through the first implant;
a second opening extending through the second implant; and
wherein the first opening communicates with the second opening.

14. The system of claim 10, further comprising a lordotic angle less than about 12° and a lordotic angle greater than about 2°.

15. The system of claim 10, wherein the system is configured to fit between vertebrae.

16. The system of claim 10, wherein at least one spinal implant comprises a metal.

17. The system of claim 10, wherein at least one spinal implant comprises a ceramic.

18. The system of claim 10, wherein at least one spinal implant comprises a polymer.

19. A method of stabilizing a human spine, comprising:
interlocking a first implant to a second implant without the use of a fastener to couple the first implant to the second implant; and
inserting the interlocked first implant and second implant between vertebrae.

20. The method of claim 19, wherein interlocking the first implant to the second implant comprises placing a protrusion on the first implant in a indentation on the second implant.
